(19) United States
(12) Patent Application Publication
(10) Pub. No.: US 2006/0184075 A1
(43) Pub. Date: Aug. 17, 2006

(54) Method and device for applying pressure waves to the body of an organism

(75) Inventors: Karl-Heinz Restle, Kreuzlingen (CH); Reiner Schultheiss, Illighausen (CH)

Correspondence Address: DUBOIS, BRYANT, CAMPBELL & SCHWARTZ, LLP, 700 LAVACA STREET, SUITE 1300, AUSTIN, TX, 78701, US

(73) Assignee: HMT HIGH MEDICAL TECHNOLOGIES AG

(21) Appl. No.: 11/386,951

(22) Filed: Mar. 22, 2006

Related U.S. Application Data
(63) Continuation of application No. 10/052,222, filed on Jan. 18, 2002.

(30) Foreign Application Priority Data
Jan. 19, 2001 (DE) 101 02 317.0

(51) Int. Cl. A61H 1/00; A61B 8/12

(52) U.S. Cl. 601/2; 600/439

(57) ABSTRACT

A method and device are described for applying extracorporeally generated pressure waves, specifically acoustic shock waves, to the body of an organism. The effect of the shock waves in the impacted target area of the body is measured by extracorporeally disposed detectors which record the acoustic signals, the acoustic signals being generated within the tissue by the cavitation bubbles caused by the shock waves. The measured cavitation effect may be utilized to control and adjust the dosage of shock waves. The use of focused detectors allows for spatial scanning of the cavitation effect—with the result that the focus of the shock waves may be controlled, the tissue structure may be scanned, and the pressure field of the shock waves may be mapped.

### CROSS-REFERENCE TO RELATED APPLICATIONS

The application claims the benefit of priority under 35 U.S.C. §119(a)-(d) from German Application No. 101 02 317.0, entitled, “Verfahren und Vorrichtung zur Beaufschlagunu des Korpers eines Lebewesens mit Druckwellen,” filed on Jan. 19, 2001 and U.S. application Ser. No. 10/052,222, entitled, “Method and Device for Applying Pressure Waves to the Body of an Organism,” filed on Jan. 18, 2002, which disclosures are incorporated herein by reference.

The invention relates to a method and device for applying extracorporeally generated acoustic pressure waves to the body of an organism.

Acoustic pressure waves are applied in medicine in various forms, for example, as ultrasound waves, as pulsed ultrasound waves, and as shock waves.

Acoustic shock waves are characterized by a short positive pressure pulse exhibiting a steep rise and high amplitude followed by a negative pressure pulse of low amplitude and of longer time. There exists in medicine the known method of applying such acoustic shock waves, for example, for destroying bodily concretions, and kidney stones in particular. Shock waves are similarly used to stimulate bone growth or to treat the tissue of soft body parts. During treatment, the shock wave dosages are generally determined empirically. Pulse energy, penetration depth, application frequency, and number of applications are generally selected based on experience. This means that the treatment requires a great amount of experience on the part of the physician—a factor which is disadvantageous for the use of such equipment. Additionally, the therapeutic success of these empirical methods is often not optimal since an excessively low dose reduces the desired success rate, while an excessively high dose can result in undesirable damage to the tissue not targeted for treatment.

The object of the invention is to provide a method and device for applying extracorporeally generated acoustic pressure waves to the body of an organism, specifically shock waves, which method and device offer a high level of control and dosing of the effect of the pressure waves.

This object is achieved according to the invention by a method having the characteristic features of Claim 1 and a device having the characteristic features of Claim 8.

Advantageous embodiments of the invention are provided in the referenced subclaims.

The invention is based on the known principle first of all that the application of pressure waves to bodily tissues, specifically shock waves, may be associated with a cavitation effect. This cavitation is produced by the fact that gas bubbles within the tissue are affected by the pressure of the shock wave. The positive pulse of the shock wave causes the gas bubbles to be compressed, while the subsequent negative pressure amplitude results in the small gas bubbles expanding and enlarging. The occurrence of such cavitation bubbles is thus an indicator of the effect of the shock wave. In addition, cavitation bubbles may also be generated by shock waves when the treated medium is inhomogeneous or contaminated. Nonhomogeneities or impurities act as seeds for cavitation.

The creation of cavitation bubbles can be detected acoustically (Cleveland, Sapozhnikof, Bailey and Crum, “A Dual Passive Cavitation Detector for Localized Detection of Lithotripsy-Induced Cavitation in Vitro,” J. Acoust. Soc. Am. 107 (3), March 2000). The cavitation bubbles generate an acoustic signal which, as a rule, occurs as a double signal, the first signal indicating the compression of small bubbles by the positive pressure pulse, and a second signal being generated with a delay when the cavitation bubbles enlarged by the negative pressure amplitude collapse again. The cavitation bubbles may be recorded and localized by acoustic detectors on the basis of these acoustic signals.

According to the invention, for treating the body of an organism, i.e., a human being or an animal, at least one acoustic detector may be disposed extracorporeally for the purpose of detecting and possibly localizing the cavitation bubbles created by application of the shock wave. By detecting the cavitation bubbles, the effect of the shock wave within the treated tissue may be recorded by measuring equipment. The physician applying the treatment is thus no longer dependent on values gained from experience to set the dosage for the shock waves, but is able to optimize the dosage individually for each treatment.

The course of treatment with pressure waves may, for example, begin with a low pulse energy level at which no cavitation so far occurs, i.e., the acoustic detectors do not yet receive any signals. The pulse energy of the shock wave or pressure wave is then increased. The adjustment of or increase in the energy level is performed based on the technique used to generate the shock waves, for example, electrohydraulic, electromagnetic, piezoelectric, or ballistic generation. When generating the shock wave by spark discharge, for example, the applied high voltage may be increased. The onset of cavitation here is determined acoustically by the detector. A further increase in the pulse energy results in a stronger cavitation effect. By acoustically monitoring the cavitation effect, the energy of the shock wave may be set to that value which on the one hand achieves the best therapeutic effect, while on the other hand avoids excessive energy producing a damaging effect without improving the therapeutic effect.

The optimum pulse parameters for the shock wave may be determined and set within one, or a very few, applications. Subsequent treatment may then be optimally administered using the shock wave parameters set by this procedure.

The method according to the invention and device according to the invention are especially well suited for automatic control. The shock wave parameters required for optimal treatment are set as the target value for the associated cavitation effect. The cavitation produced by the shock waves is then measured by the extracorporeal detector as the actual value and the shock wave parameters are automatically adjusted to set the measured actual value of cavitation to match the specified target value.

Based on the measurement of the shock wave effect of cavitation, the desired treatment may be performed in an optimal manner with no physician experience or physician intervention being necessary. The shock wave or pressure wave, i.e., the energy, pulse shape, pulse sequence, rise time, tension component, etc. of the wave, or the focus position as well, must simply be adjusted, either manually or by automatic control, such that the measured cavitation matches the specified value. Since the shock wave generation occurs within the treatment target area in accordance with the actual effect really measured, the result is that: differences in tissue structure from patient to patient are automatically taken into account; varying attenuations of the shock waves as they pass through the body to the target area, for example due to the tissue structures traversed, tissue thickness, etc. are compensated; changes in the tissue structure, for example, due to the respiratory movements of the patient, are taken into account; and finally, even short-term changes in the tissue structure, for example, due to the effect of the shock waves themselves are compensated.

The acoustic measurement of the effect of the shock waves within the target area may also be exploited in other ways. The dependence of the cavitation bubble formation on the tissue structure may, for example, be exploited to analyze the tissue structure, composition or differentiation by means of predetermined shock waves.

If pressure waves of predetermined energy levels are introduced into the body, the interfaces between the various tissue materials may be determined based on the changing cavitation effect at this interface. This factor may, for example, be advantageously exploited when shock waves are applied to bone in order to stimulate bone growth. The discontinuous changes in the cavitation effect at the bone surface allow for a precise focusing or positioning of the shock wave, or detection of the interface.

In addition, the tissue structure may be scanned over a greater spatial area to obtain an image of the tissue anatomy. To accomplish this, a pressure wave with a predetermined parameters may be applied to a larger target area, and the cavitation bubble formation which changes locally according to the varying tissue structure may be differentially scanned using focused detectors.

The reverse procedure is also possible in which the spatial pressure field of the pressure wave is determined and displayed within a known tissue structure of the target area based on the measured spatial distribution of the cavitation effect, for example, as a means for determining and controlling the focus of the shock wave source.

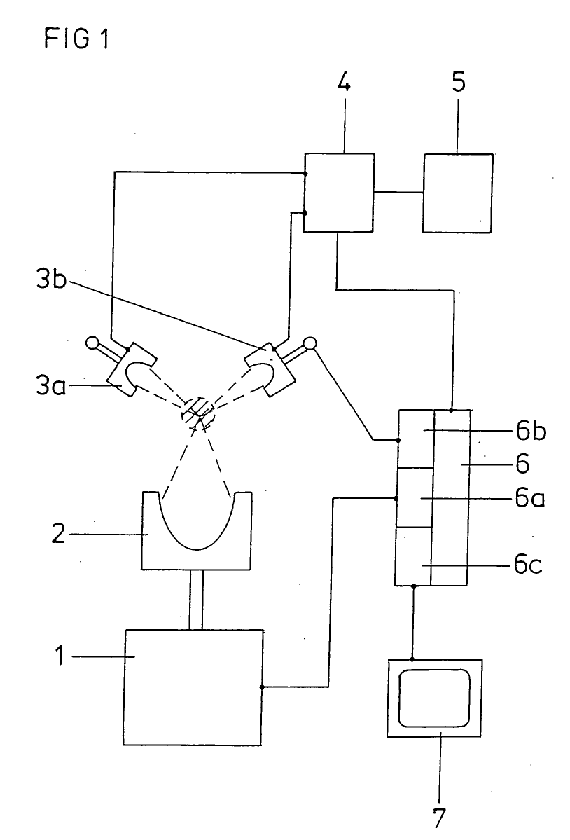

The following discussion explains the invention in greater detail based on several embodiments. FIG. 1 shows a device according to the invention in schematic form.

FIG. 1 shows a shock wave generator 1 with a treatment head 2. The shock wave generator 1 contains the familiar power and voltage supply together with the associated control electronics. Treatment head 2 is a familiar pressure wave or shock wave generator and has, for example, a volume of liquid with a shock wave source consisting, for example, of two high-voltage electrodes, piezoelements, etc. Treatment head 2 is placed on the surface of the body of the human or animal subject undergoing treatment and can inject the shock waves generated in treatment head 2 into the body and focus them in a target area inside the body.

In addition, the device has at least one acoustic detector, preferably two detectors, which are identified as 3a and 3b. Additional analogously designed detectors may be used as required. Detectors 3a, 3b are microphones or hydrophones which are placed preferably extracorporeally on the body surface. Detectors 3a, 3b are preferably focusable so that they may receive directed acoustic signals from a defined target area

The acoustic signals received from detectors 3a, 3b are converted by detectors 3a, 3b into electrical signals which are fed to an electronic evaluation means 4. When two or more detectors 3a, 3b are employed, the electronic evaluation means contains specifically a coincidence device which assigns the signals received from detectors 3a, 3b to the same event, i.e., to the same cavitation bubbles. The overall function of electronic evaluation means 4 is to qualify the measured cavitation effect and, for example, to indicate the location, size, lifetime, quantity and/or density of the cavitation bubbles. The signals analyzed in electronic evaluation means 4 are displayed in a display unit 5. Presentation of the signals in display unit 5 may be accomplished in different ways. The simplest type of display consists of a luminous indicator which shows simply whether or not acoustic signals are being received. A more informative display may consist of three display lamps which indicate respectively whether the effect of the shock wave introduced by treatment head 2 into the target area lies below, at, or above the cavitation threshold. It is also possible to equip display unit 5 with an analog display, for example, a meter or a light-strip indicator which displays quantitatively the acoustic signals of the cavitation bubbles received by detectors 3a, 3b.

The signals processed by electronic evaluation means 4 are also fed to a feedback system 6 which may be provided in addition to display unit 5 or which may completely replace display unit 5.

Feedback system 6 may perform the following functions which may be provided either in combined form or as alternatives. Feedback system 6 may act on shock wave generator 1 through automatic control 6a to control the adjustment parameters for treatment head 2 such that the actual value of cavitation measured by detectors 3a, 3b is adjusted to match a specified target value. In addition, feedback system 6 may use an actuating signal generator 6b to generate actuating signals for the alignment mechanism of detectors 3a, 3b. Finally, feedback system 6 may generate data for an image-processing system 7 via an image generator 6c.

The device according to the invention offers the following possible applications.

When a certain region of a patient's body is to be treated with shock waves, treatment head 2 is placed on the patient's body and focused on the target area. Detectors 3a, 3b are similarly placed on the surface of the body and focused on this target area. When shock wave generator 1 is operated, detectors 3a, 3b measure the effect of the shock waves produced by the cavitation bubbles in the target area. The effect of the shock waves in the target area is displayed by display unit 5. Based on the information indicated by display 5, the operators may adjust shock wave generator 1 to achieve the desired shock wave effect within the target area. When feedback system 6 is used, a target value for the shock wave effect may be specified for control unit 6a, which value is automatically adjusted by shock wave generator 1. In this way, the shock wave treatment of the target area may be performed, for example according to the principle “as much as necessary, as little as possible.”

When a defined, locally limited target area is to be treated with shock waves, the shock waves emitted by treatment head 2 are focused on this target area. As a result, the effect of the shock waves and the formation of cavitation bubbles is accordingly the strongest in this target area. Since the cavitation bubbles are thus first created in this target area as the shock wave energy increases, a single detector 3 may be sufficient for a single measurement, although this detector 3 does not need to be focused on the target area. A simple determination of the cavitation threshold within the target area may be performed by a single integrally measuring, unfocussed detector 3.

The employment of focused detectors 3 and of two or more detectors 3a, 3b additionally allows for a more precise spatial measurement of the cavitation. In this approach, noise signals may be blanked out. When there is a higher dosage of shock waves resulting in a stronger propagation of the cavitation effect, the shock wave effect may be measured within a specific target area. Similarly, the spatial distribution of the shock wave effect may be determined by focused detectors 3a, 3b.

A measurement of coincidence using two focused detectors 3a, 3b makes it possible to localize spatially the initiation site of the acoustic signals within a volume with a diameter of 0.2 mm to 20 mm. As a result, it is possible to differentially scan the cavitation bubbles created by the shock waves in terms of their spatial distribution and intensity as well. To achieve this, detectors 3a, 3b may be moved three-dimensionally and aligned—a task which may, as appropriate, be accomplished by actuating signal generator 6b of feedback system 6. Using image generator 6c of feedback system 6 within image-processing system 7, the spatial distribution and intensity of the created cavitation bubbles measured by this spatial scanning procedure may be displayed three-dimensionally on a monitor and/or recorded.

Measurement of the spatial distribution and intensity of the generated cavitation bubbles by the focused detectors 3a, 3b, and by possible additional detectors within a coincidence circuit, provides for the following additional applications.

When the shock wave field in the bodily tissue is scanned differentially and three-dimensionally by detectors 3a, 3b, differences in the tissue structure may be determined based on the changes in the cavitation effect. Specifically, interfaces between the different tissue materials may be determined which are associated with a discontinuity in the impedance of the transmitted shock waves and with an increased shock wave reflection. This feature may, for example, be utilized to determine the surface of a bone undergoing treatment, a calcium deposit to be destroyed, or a bodily concretion so that the shock waves may be focused or precisely positioned on this target area.

In addition, the anatomical tissue structure within a larger spatial region may be mapped and imaged as required. To achieve this, the shock wave field generated by treatment head 2 and the focus area of detectors 3a, 3b are simultaneously displaced. The fact that the measured cavitation signal in response to an identical shock wave effect depends on the character of the specific targeted tissue allows a three-dimensional graphic representation of the tissue structure to be obtained. A corresponding determination of the tissue structure may be obtained by measuring the cavitation threshold of the shock wave energy level at which cavitation begins for each target point of the three-dimensional region.

An additional possibility is to use focused detectors 3a, 3b to map the shock wave field generated by treatment head 2 three-dimensionally. Given a known tissue structure which has, for example, been measured by ultrasound, the shock wave field caused by cavitation is mapped three-dimensionally. Based on the known spatial distribution of the tissue structure and the measured cavitation, the spatial distribution of the shock wave effect, and thus the spatial pressure field, may be computationally analyzed and displayed as required.

### LIST OF REFERENCE NUMBERS

1 Shock wave generator
2 Treatment head
3a/3b Detectors
4 Electronic evaluation means
5 Display unit
6 Feedback system
6a Control unit
6b Actuating signal generator
6c Image generator
7 Image-processing system

1. Method for applying extracorporeally generated acoustic pressure waves, specifically shock waves, to the body of an organism,
characterized in that the effect of the pressure waves within the treated target area is determined using cavitation bubbles generated within the tissue of the body by recording the acoustic signals of these bubbles using at least one preferably extracorporeally disposed detector.

2. Method according to claim 1,
characterized in that the acoustic signals are employed to measure an actual value for the effect of the pressure waves in a selected target area and the parameters of the generated pressure waves are adjusted such that the effect of the pressure waves within the target area reaches a predetermined target value.

3. Method according to claim 2,
characterized in that the parameters of the pressure waves generated are adjusted by an automatic control.

4. Method according to claim 1,
characterized in that the effect of the pressure waves is spatially scanned in the target area of the body using the cavitation effect measured by at least one focused detector.

5. Method according to claim 4,
characterized in that the local change in the measured cavitation effect is evaluated to determine the interface between different tissue materials.

6. Method according to claim 4,
characterized in that the local change in the measured cavitation effect is evaluated to determine the spatial tissue anatomy.

7. Method according to claim 1,
characterized in that the spatial change in the cavitation effect is scanned by at least one focused detector and that the spatial pressure field of the pressure waves is computed from the measured spatial distribution of the cavitation effect and the known tissue structure.

8. Device for applying extracorporeally generated acoustic pressure waves to the body of an organism including a pressure wave generator (1) and a treatment head (2),
characterized by: at least one acoustic detector (3a, 3b) brought in contact with the surface of the body to record the acoustic signals from the cavitation bubbles generated by the pressure waves and characterized by an electronic evaluation means (4) to which the signals from at least one detector (3a, 3b) are: fed, the parameters for the pressure waves generated by the pressure wave generator (1) being adjusted in response to the signals processed in the electronic evaluation means (4).

9. Device according to claim 8,
characterized in that at least one detector (3a, 3b) is focusable.

10. Device according to claim 9,
characterized in that at least two detectors (3a, 3b) are connected in a coincidence circuit.

11. Device according to claim 10,
characterized in that the focus area of the at least one focused detector (3a, 3b) is spatially adjustable for scanning a target area of the body.

12. Device according to claim 11,
characterized in that the electronic evaluation means (4) controls a display unit (5) which displays the measured cavitation effect.

13. Device according to claim 12,
characterized in that the electronic evaluation means (4) drives a feedback system (6).

14. Device according to claim 13,
characterized in that the feedback system (6) includes a control unit (6a) which controls the pressure wave generator (1) such that the actual value of the cavitation effect determined by the at least one detector (3a, 3b) and the electronic evaluation means (4) is adjusted to match a predetermined target value.

15. Device according to claim 13,
characterized in that the feedback system (6) includes an actuating signal generator (6b) which controls the spatial adjustment of the at least one detector (3a, 3b).

16. Device according to claim 13,
characterized in that the feedback system (6) includes an image generator (6c) which feeds the data generated by the electronic evaluation means (4) to an image-processing system (7).
